(19) United States
(12) Patent Application Publication
(10) Pub. No.: US 2011/0082368 A1
(43) Pub. Date: Apr. 7, 2011

(54) RECONSTRUCTION OF DYNAMICAL CARDIAC SPECT FOR MEASURING TRACER UPTAKE AND REDISTRIBUTION

(75) Inventor: Henrik Botterweck, Aachen (DE)

(73) Assignee: KONINKLIJKE PHILIPS ELECTRONICS N.V., EINDHOVEN (NL)

(21) Appl. No.: 12/993,991

(22) PCT Filed: May 29, 2009
(86) PCT No.: PCT/IB2009/052282

§ 371 Date: Nov. 22, 2010

Related U.S. Application Data
(60) Provisional application No. 61/058,579, filed on Jun. 4, 2008.

(51) Int. Cl. A61B 5/05; G06K 9/00; G01T 1/166

(52) U.S. Cl. 600/425; 382/131; 250/363.04

(57) ABSTRACT

When performing a static image reconstruction of acquired single photon emission computed tomography (SPECT) data for myocardium, dynamic tracer uptake, redistribution, and washout information is generated with reduced or eliminated artifacts by back-projecting ray projections onto a reconstructed myocardial surface. A complete SPECT scan is performed after tracer injection, and a static image of the myocardial surface is reconstructed. The reconstructed image is segmented and a polar plot of it is generated. A contemporaneously acquired subset of the SPECT projection data is then back-projected onto the segmented surface of the polar plot. Contributions from emissions not originating from the myocardium (e.g., from adjacent anatomical structures) are compensated. The resultant image data, which describes tracer distributions across heart segments per projection time, are overlaid on the polar plot and presented to a user. In this manner, time-dependent tracer perfusion is supplied to the user despite the static nature of SPECT imaging systems.

The present innovation finds particular application in patient imaging systems, particularly involving patient imaging devices such as single photon emission computed tomography (SPECT) and the like. However, it will be appreciated that the described technique may also find application in other nuclear imaging systems, other patient imaging scenarios, other image analysis techniques, and the like.

In SPECT imaging systems, a planar detector collects projection data to accumulate a sufficient amount of data. In combined SPECT-computed tomography (CT) systems, typically one to three detectors are stepped to each of a plurality of positions to collect sufficient data along each of a plurality of projection directions for the reconstruction of a diagnostically meaningful 3D image. Tracer uptake, perfusion, and washout dynamics are not typically analyzed, since SPECT is non-dynamic system. Tracer distribution can be adversely affected by, for example, initial uptake by the liver, reduced uptake and washout rates associated with ischemic cardiac tissue, etc., which can lead to unwanted artifacts. These uncontrolled effects degrade a reconstructed SPECT image.

There are two main reasons for a time-varying tracer distribution during a cardiac SPECT acquisition: if aiming for a short work-flow (e.g. a fast rest-stress systolic dyssynchrony index protocol), an initial uptake dynamic may occur combined with signals from the blood-pool and liver. Additionally, in the case of thallium-chloride, ischemic regions show a retarded uptake under stress conditions and slower wash-out in the subsequent rest-phase. Such effects are unwanted during SPECT acquisitions, because they lead to artifacts in the reconstructed volume image. They may be corrected for by preprocessing the measured projections, i.e. by filtering and normalizing along sinogram lines.

Uncontrolled uptake effects after injection and/or re-distribution and wash-out can degrade the reconstructed image quality considerably. On the other hand, a quantitative measure for these gradient “speeds” may help in classifying ischemic, “hibernating,” and infarcted tissue. There are methods for dynamic SPECT reconstruction, but they lack in general stability and robustness, mainly due to the even more severely ill-posed reconstruction problem in this case.

Time-dependent processes in SPECT are a problem due to its inherently static data acquisition, as compared, e.g., with positron emission tomography (PET). At one point in time, there are typically one or two projection views of the region of interest collected. Different projection directions often represent projections of different radioisotope distributions due to the time processes, which leads to artifacts in 3D images. In the case of cardiac acquisitions, for example, the clinical decision is typically done by assessing the perfusion as shown in a polar plot, e.g., a two-dimensional projection of the left chamber myocardium.

The present application provides new and improved systems and methods for estimating a time-dependent polar plot of tracer distribution during a SPECT acquisition, which overcome the above-referenced problems and others.

In accordance with one aspect, an artifact correction system for tracer uptake images includes a processor that receives a plurality of tracer uptake projection data sets from a region of interest, statically reconstructs an image of the region of interest, generates a polar plot of the surface of the region of interest, and back-projects a temporally limited segment of the uptake projection data from the static reconstruction of the image onto the polar plot of the surface of the region of interest.

In accordance with another aspect, a method of generating dynamic cardiac single photon emission computed tomography (SPECT) images includes reconstructing a three-dimensional image including the region of interest, segmenting the region of interest from the three-dimensional image, and generating a polar plot image of a surface of the region of interest. The method further includes back-projecting a contemporaneously collected segment of SPECT projection data onto the polar plot image, and outputting to a user the polar plot image of the surface of the region of interest overlaid with tracer distributions from the SPECT data.

In accordance with another aspect, an apparatus for generating dynamic cardiac single photon emission computed tomography (SPECT) images includes means for performing a SPECT data acquisition on a region of interest after tracer injection, means for reconstructing a three-dimensional image including a region of interest, and means for segmenting the region of interest from the three-dimensional image. The apparatus further includes means for generating a polar plot image of a surface of the region of interest, means back-projecting a contemporaneously collected segment of SPECT projection data onto the polar plot image, and means for outputting to a user the polar plot image of the surface of the region of interest overlaid with tracer distributions from the SPECT data.

One advantage is that time dependent tracer uptake information is used to reduce artifacts.

Another advantage resides in visibility of time-dependent changes to a user.

Still further advantages of the subject innovation will be appreciated by those of ordinary skill in the art upon reading and understand the following detailed description.

The innovation may take form in various components and arrangements of components, and in various steps and arrangements of steps. The drawings are only for purposes of illustrating various aspects and are not to be construed as limiting the invention.

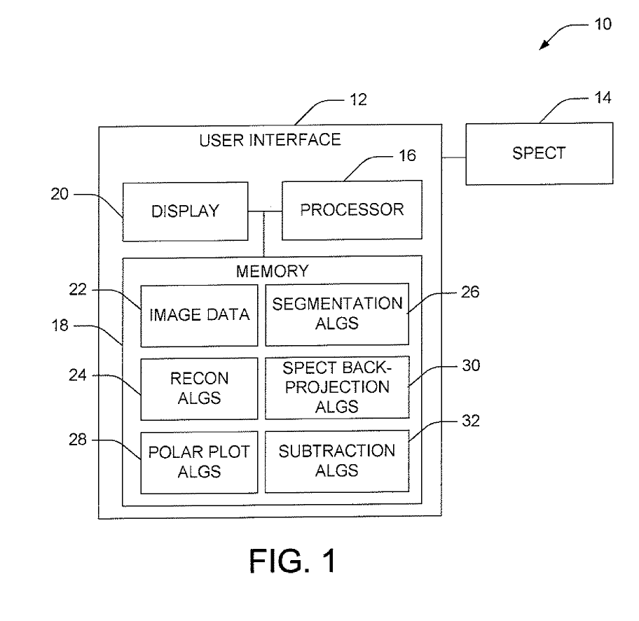

FIG. 1 illustrates a system that uses polar-plot reconstruction of SPECT images to correct tracer uptake and redistribution data.

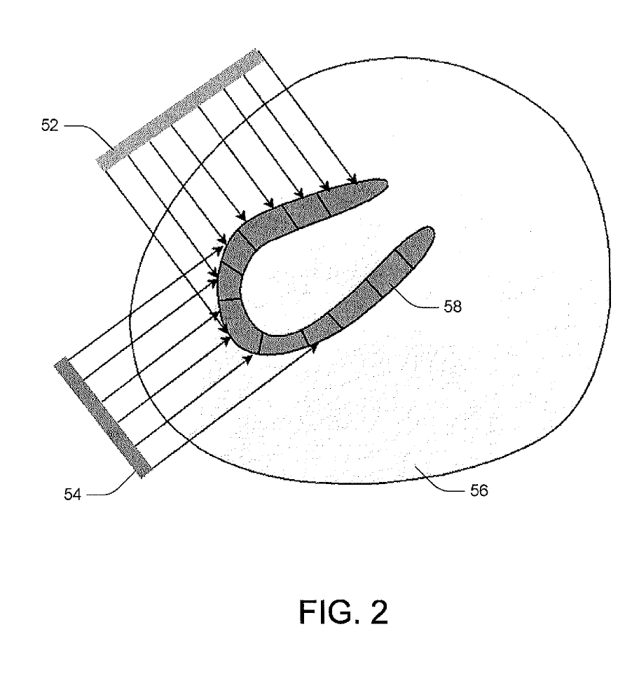

FIG. 2 illustrates a camera pair of projection data sets arranged in a substantially orthogonal orientation relative to each other to project rays toward a volume of interest.

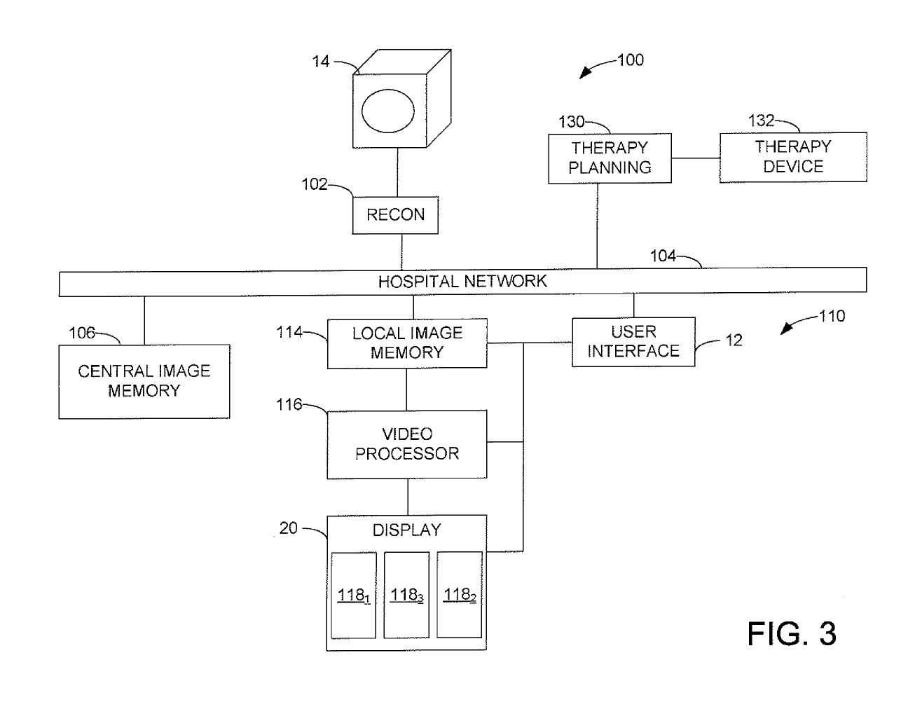

FIG. 3 illustrates an exemplary hospital system that may include an imaging device, such as a SPECT imaging device, or the like, which generates imaging data that are reconstructed by one or more reconstruction processors to generate 3D image representations.

FIG. 1 illustrates a system 10 that uses polar-plot reconstruction of SPECT images to correct tracer uptake and redistribution data. The system performs time-dependent reconstruction by back-projecting original SPECT projections onto a known surface of a heart (or other anatomical structure, such as an organ, tumor, etc.). In one embodiment, a cardiac SPECT acquisition is performed, and attenuation data is collected from transmission measurements from a line source or CT. A simple reconstruction (e.g., 1-2 iterations) is performed, and the heart is located and segmented to generate a polar plot. Once the heart surface is known or identified, the original SPECT projections are back-projected thereon. Non-cardiac emissions are then subtracted from the image, and can be estimated from a static reconstruction performed prior to generating a polar plot of the heart or a portion thereof. Tracer distributions across heart segments per unit of projection time are then presented as a gradient overlaid on the static polar plot or a 3D image.

The system 10 includes a user interface 12 that receives SPECT data, in the form of projection data sets, acquired by a SPECT imaging device 14 during a scan of a subject over a plurality of projection directions. In one embodiment, the SPECT device includes a projection region source, e.g., a line source, which causes the SPECT detectors to generate an attenuation data set concurrently with the SPECT data set. The user interface includes a processor 16 that executes, and a memory 18 that stores, acquired SPECT data, attenuation data, and other relevant image data, and a plurality of computer-executable algorithms for carrying out the various procedures and functions described herein. Information is output to a user via a display 20.

In one embodiment, the processor 18 receives acquired SPECT data from the SPECT device, and attenuation data from the SPECT device, a CT scanner, or the like. The processor generates image data by executing reconstruction algorithm(s) 24 on the acquired SPECT and/or attenuation data to generate a static SPECT reconstructed image and/or a 3D attenuation image or a combination of the two. The image(s) is then segmented and a polar plot of the heart is generated using segmentation algorithm(s) 26 and polar plot algorithm(s) 28, respectively. In an additional reconstruction step, the individual projections used during the original SPECT static image reconstruction are backprojected onto the heart surface, which is known from the polar plot, by executing SPECT back-projection algorithm(s) 30. The processor executes subtraction algorithm(s) 32 to subtract emissions from outside of the heart to leave an image of just the heart surface with the SPECT data projected thereon. In one embodiment, emissions from outside the heart are estimated from the static reconstruction, which is performed by executing the reconstruction algorithm(s) 24. By back-projecting SPECT projection data sets that were acquired at different times, a time-dependent polar plot of tracer distribution during SPECT acquisition is generated. In one embodiment, the SPECT data is collected in a list mode, i.e., each received SPECT radiation event is time-stamped. This permits temporal resolution of the data within a single projection data set. Moreover, temporal windows can be defined that span parts of two or more projection data sets.

The dynamics of tracer uptake, re-distribution and wash-out for cardiac SPECT imaging gives relevant information, but conventionally has been considered a source of reconstruction problems due to the inherent non-dynamic nature of SPECT. The systems and methods described herein estimate the time-dependent polar-plot of tracer distribution during a SPECT acquisition. This additional information is used to correct for artifacts, such as occur due to acquisitions early after a technetium-99m (Tc-99m) injection, or for clinical evaluation, such as is related to the thallium-201 (Tl-201) speed of re-distribution and wash-out. The procedure is based on a raw heart segmentation (which may be combined with an automatic segmentation method developed) and uses an examined image (e.g., the polar-plot), which is effectively two-dimensional. Moreover, the procedure can be performed as part of a standard reconstruction and hence does not disturb the usual workflow. The described systems and methods can be combined with simultaneous transmission measurements to correct the time-dependent data and heart registration for patient movements and/or breathing. A generalization to other applications (e.g., cardiac or oncology with localized hot-spots) is also contemplated.

Obtained segmented perfusion values at a given time are used in addition to the 3D-reconstructed data and are used to correct them and/or give additional information on time dependencies. In one embodiment, it is assumed that the myocardium is essentially two-dimensional, at least with respect to perfusion evaluation, and that emissions outside the heart are either weak (e.g., as in the lungs) or of a known timely behavior (e.g., as in the liver), so that they may be taken into account when performing the backprojection. This results in a quantitative estimation of the time variation of the myocardium perfusion.

With reference to FIG. 2, and with continuing reference to FIG. 1, during data acquisition, two radiation detectors or camera heads are positioned in an approximately 90° orientation relative to each other to collect concurrently a first projection SPECT data set 52 and a second projection SPECT data set 54 that are arranged in a substantially orthogonal orientation relative to each other. A normal cardiac SPECT acquisition is performed by stepping the orthogonally positioned detectors or camera heads around the region of interest. For instance, a stress exercise (ergometer) can be carried out until cardiac stress of a subject is nearly maximal before tracer injection in order to obtain good differential information on tracer uptake and distribution compared to a rest image. The detectors or camera heads are in an approximately 90 degree orientation relative to each other, so that each projection ray intersects the myocardium at one or two (anterior/posterior) positions. Attenuation data is obtained from transmission measurements (e.g., CT or line source) by the processor 16.

It will be appreciated that although the system is described as having two detectors or camera heads arranged at approximately a 90 degree angle to each other, N detectors or camera heads, where N is an integer, may be used in different orientations that are not limited to orthogonal arrangements.

The processor then performs a normal static reconstruction, for instance by executing reconstruction algorithm(s) 24. In one embodiment, a few iterations of a statistical algorithm, such as an ordered subset expectation maximization (OSEM) algorithm or a filtered back-projection (FBP) reconstruction algorithm are sufficient. After reconstruction, the heart is located and segmented for polar-plot purposes. The segmentation is either semi-automated, as is performed using, e.g., AutoQuant+, or fully automatic with a tool such as an automatic heart-segmentation tool. The latter may be adapted and simplified for this purpose. In one embodiment, the foregoing is performed by the processor 16 by executing segmentation algorithm(s) 26 and polar plot algorithm(s) 28, respectively.

In an additional reconstruction step, the processor executes back-projection algorithm(s) 30, to back-project orthogonal pairs of contemporaneous projections onto the now known heart surface. Any ambiguity between anterior and posterior intersections of a ray with the left myocardium is resolved by the orthogonal view provided by the other camera. Two non-parallel projections are sufficient for the reconstruction of a two-dimensional object. The processor executes subtraction algorithm(s) 32 to subtract or segment emissions originating outside the heart from the image. Such emissions are estimated from the static reconstruction.

Attenuation and scatter are compensated in the same way as for the 3D-reconstruction, e.g. by scaling the projection ray accordingly. Monte-Carlo or effective source scatter estimation (ESSE) type correction data is already calculated in the static reconstruction and can be re-used. The results are tracer-distributions across the heart segments per projection time. They may be presented on the display 20 as a gradient overlaid to the static polar-plot or 3D-image, or may be used for additional post-processing. The process can be repeated for a plurality of the orthogonal projection data pairs and the overlaid polar plots can be displayed sequentially, e.g., in a cine type display, to show the time evolution of the tracer distributions.

According to another embodiment, when time dependent information on the position, heart registration and attenuation of the patient is available, the projections can be backprojected using a heart registration and attenuation map optimized from the simultaneously obtained transmission projection. That is, patient motion may be corrected by matching the transmission projection with the 3D-transmission reconstruction/attenuation map from the whole data. Additionally or alternatively, a systolic dyssynchrony index (SDI) acquisition can be performed with simultaneous measurement of Tc-99m and Tl-201 in an analog manner Other nuclides such as Tc-99m and iodine-123 (I-123) can be also used. Moreover, other objects can be imaged, as long as their emission distribution is less than three-dimensional. This holds for quantitative oncology applications, where tumors are either small hot spots (e.g., point-like) or, when extended, their absolute integrated emission is of interest. In the latter case, an initial 3D reconstruction is performed for detection, classification, and delineation of a region of interest (ROI). In the classification step, the time-dependent absolute tracer uptake is estimated as before, from the projections.

For a small ROI, the nuclear camera detectors or heads can be arranged in a 180-degree orientation. Time dependencies over longer periods of time can be measured without successive full scans. For example, after the initial acquisition with a full 180/360 degree gantry rotation, single projections may be obtained to be processed with the earlier determined ROI, with the portions of interest (e.g., lesions) are in the field of view.

According to other embodiments, the described systems and methods are used for the evaluation of SPECT acquisitions. Although the main embodiment is described with regard to cardiologic applications, it will be appreciated that other applications are contemplated and that the described systems and methods are not limited thereto. It is suited to SPECT systems with simultaneous transmission for time-dependent registration and attenuation estimation. Alternatively, standard SPECT/CT or transmission source systems can be used. Moreover, the system 10 can be used to correct standard reconstructions, for instance in a fast work-flow where reconstruction is performed after tracer injection. Furthermore, the system can be used for obtaining additional dynamic information related to tracer uptake and re-distribution (e.g., for Tl-201, Tc-99m, or 1-123).

With reference to FIG. 3, an exemplary hospital system 100 may include an imaging device, such as a SPECT imaging device 14, or the like, which generates imaging data that are reconstructed by one or more reconstruction processors 102 to generate 3D image representations. The image representations are communicated over a network 104 to a central memory 106 or a local memory 114.

At a station 110 connected with the network, an operator uses the user interface 12 to move a selected 3D patient image representation to or between the central memory 106 and the local memory 114. A video processor 116 displays the selected patient image representation in a first viewport 118₁, of the display 20. Tracer distributions are displayed in a second viewport 118₂. A third view port 118₃ can display an overlay of the tracer distributions and the image representation. For example, a user can be permitted to register landmarks in a tracer distribution image to corresponding structures or landmarks in a polar plot image. For instance, the operator, through the interface 12, selects the polar plot image landmarks (e.g., using a mouse, stylus, or other suitable user input device) that correspond to landmarks in the tracer uptake image. Alternately, the tracer uptake can be aligned automatically by a program in the processor 116. The processor 16 (FIG. 1) in the user interface 12 then performs correction algorithms and infers an appropriate tissue type to employ when filling in truncated areas in the attenuation map.

The overlay image can then be used in other applications. For instance, a therapy planning station 130 can use the overlay image to plan a therapy session. Once planned to the satisfaction of the operator, the planned therapy can, where appropriate to an automated procedure, be transferred to a therapy device 132 that implements the planned session. Other stations may use the overlay image in various other planning processes.

In another embodiment, the overlay displayed in viewport 118₃ is adjustable to weight the patient image data relative to the tracer uptake image, or vice versa. For instance a slider bar or knob (not shown), which may be mechanical or presented on the display 20 and manipulated with an input device, may be adjusted to vary the weight of the patient image or the tracer uptake image. In one example, an operator can adjust the image in viewport 118₃ from purely patient (polar plot) image data (as is shown in viewport 118₁), through multiple and/or continuous combinations of patient and tracer uptake image data, to purely tracer uptake image data (as is shown in viewport 118₂). For instance, a ratio of patient image data to tracer uptake image data can be discretely or continuously adjusted from 0:1 to 1:0.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. An artifact correction system for tracer uptake images, including:
a processor that receives a plurality of tracer uptake projection data sets from a region of interest, statically reconstructs an image of the region of interest, generates a polar plot of the surface of the region of interest, and back-projects a temporally limited segment of the uptake projection data from the static reconstruction of the image onto the polar plot of the surface of the region of interest.

2. The system according to claim 1, further including a single photon emission computed tomography (SPECT) scanner with camera heads arranged at approximately a 90 degree orientation relative to each other to generate pairs of contemporaneous projection data sets, so that each of a plurality of projection rays emitted from one of the pairs of contemporaneous projection data sets intersects the surface of the region of interest at one or two locations.

3. The system according to claim 2, wherein the processor executes a reconstruction algorithm to generate an attenuation-corrected static reconstruction image.

4. The system according to claim 3, wherein the processor executes a segmentation algorithm to segment the surface of the region of interest and executes a polar plot algorithm to generate the polar plot of the surface of the regions of interest.

5. The system according to claim 2, wherein the processor resolves ambiguity between anterior and posterior intersections of a projection ray from a first of a contemporaneous pair of projection data sets using a substantially orthogonal projection ray from a second of the contemporaneous pair of projection data sets, and resolves ambiguity between anterior and posterior intersections of a projection ray from the second of the contemporaneous pair of projection data sets using a substantially orthogonal projection ray from the first of the contemporaneous pair of projection data sets.

6. The system according to claim 2, wherein the processor executes a subtraction algorithm to subtract emissions not originating in the region of interest from the static reconstruction image.

7. The system according to claim 2, further including a display on which tracer distributions across segments of the region of interest are overlaid on the polar plot of the surface of the region of interest.

8. The system according to claim 1, wherein the region of interest is at least one of myocardium or tumor tissue.

9. The system according to claim 1, further including a memory that stores one or more of the acquired tracer uptake projection data sets, reconstructed image data, reconstruction algorithms, segmentation algorithms, polar plot algorithms, back-projection algorithms, and subtraction algorithms for execution by the processor.

10. A method of generating dynamic cardiac single photon emission computed tomography (SPECT) images, including:
reconstructing a three-dimensional image including the region of interest;
segmenting the region of interest from the three-dimensional image;
generating a polar plot image of a surface of the region of interest;
back-projecting a contemporaneously collected segment of SPECT projection data onto the polar plot image; and
outputting to a user the polar plot image of the surface of the region of interest overlaid with tracer distributions from the SPECT data.

11. The method according to claim 10, further including, in back-projecting the contemporaneously collected segment of the SPECT projection data, compensating for emissions that did not originate in the region of interest.

12. The method according to claim 10, further including receiving attenuation data obtained from transmission measurements, and wherein the step of reconstructing the three-dimensional image includes reconstructing one or both of the attenuation data and the SPECT data.

13. The method according to claim 10, further including contemporaneously acquiring pairs of SPECT data sets including a first SPECT projection data set and a second SPECT projection data set at an approximately 90 degree orientation relative to each other, projection rays from each of the SPECT projection data sets intersecting the surface of the region of interest at one or two locations.

14. The method according to claim 13, further including resolving ambiguity between anterior and posterior intersections of a projection ray from the first SPECT projection data set using a substantially orthogonal projection ray from the second SPECT projection data set, and resolving ambiguity between anterior and posterior intersections of a projection ray from the second SPECT projection data set using a substantially orthogonal projection ray from the first SPECT projection data set.

15. The method according to claim 10, wherein the region of interest includes myocardium.

16. A dynamic tracer uptake imaging system including a processor programmed to perform the method according to claim 10.

17. A computer-readable medium having stored thereon software for controlling one or more computers to perform the method according to claim 10.

18. An apparatus for generating dynamic cardiac single photon emission computed tomography (SPECT) images, including:
means for performing a SPECT data acquisition on a region of interest after tracer injection;
means for reconstructing a three-dimensional image including a region of interest;
means for segmenting the region of interest from the three-dimensional image;
means for generating a polar plot image of a surface of the region of interest;
means back-projecting a contemporaneously collected segment of SPECT projection data onto the polar plot image; and
means for outputting to a user the polar plot image of the surface of the region of interest overlaid with tracer distributions from the SPECT data.

19. The apparatus according to claim 18, further including means for compensating for emissions that did not originate in the region of interest.
